(19) United States
(12) Patent Application Publication
(10) Pub. No.: US 2006/0205094 A1
(43) Pub. Date: Sep. 14, 2006

(54) Methods using novel chemiluminescent labels

(75) Inventors: Hashem Akhavan-Tafti, Howell, MI (US); Wenhua Xie, Novi, MI (US)

Correspondence Address: LUMIGEN, INC., 22900 W. EIGHT MILE ROAD, SOUTHFIELD, MI, 48034, US

(73) Assignee: Lumigen, Inc.

(21) Appl. No.: 11/079,899

(22) Filed: Mar. 14, 2005

(51) Int. Cl. G01N 33/533

(52) U.S. Cl. 436/546

(57) ABSTRACT

Methods using chemiluminescent label compounds and chemiluminescent labeled conjugates are provided. The compounds comprise an acridan ring bearing an exocyclic ketene dithioacetal group and further contain a labeling substituent which permits attachment to compounds of interest. The novel chemiluminescent compounds and labeled conjugates are convenient to prepare, are highly stable, and generate chemiluminescence rapidly on demand. The compounds and conjugates are useful in assays of an analyte in a sample and in assays employing labeled specific binding pairs.

### FIELD OF THE INVENTION

The present invention relates to a new method of rapidly producing chemiluminescence from electron-rich alkenes by a simple chemical process using inexpensive, readily available reagents. The present invention relates further to chemiluminescent labeling compounds, their use in preparing chemiluminescent labeled compounds and the use of the labeled compounds in assay methods. The invention further relates to assay methods for detecting an analyte and for detecting chemiluminescent-labeled analyte, especially within an electrophoresis gel. The methods are useful in immunoassays, nucleic acid probe assays and the like.

### BACKGROUND OF THE INVENTION

Chemiluminescent detection of analytes has assumed increasing importance in a number of fields, including biomedical analysis, food testing, pathogen identification, forensic investigations and environmental contaminant screening. The means of incorporating a chemiluminescent endpoint into a test or assay can take different forms, such as a chemiluminescent substrate for an enzyme label, a chemiluminescent compound shielded within a structure such as a micelle, liposome or latex particle or by using a chemiluminescent compound as a label. Numerous compounds have been devised for these purposes (R. Handley, H. Akhavan-Tafti, A. P. Schaap, J. Clin. Ligand Assay, 20(4) 302-312 (1997)). The use of chemiluminescent compounds to label species to be detected with small molecules has attracted interest due to the ability to attach multiple labels and to generate the chemiluminescence rapidly. Nevertheless, no single labeling and detection scheme has proven superior in all applications.

Chemiluminescent Labels Luminol, isoluminol and related cyclic diacyl hydrazides were the first chemiluminescent compounds to be adapted as direct labels by modifying their structure to include a linking substituent. Their use is not satisfactory for many applications due to insufficient light generation limiting detection sensitivity. The low chemiluminescence quantum efficiency, 0.1-1%, and times as long as several minutes for all of the photons to be emitted diminish instantaneous light intensity.

Acridinium esters and acridinium sulfonamides have been used extensively in chemiluminescent immunoassays. (See, e.g., U.S. Pat. No. 5,656,500, U.S. Pat. No. 5,521,103 and references cited therein). The principal advantages of these labels are the high yield of chemiluminescence (ca. 10%) coupled with the short duration of emission, typically 1-2 sec. Liberating the light energy in such a short flash creates high light intensities. The use of these labels, however, suffers from certain serious drawbacks. Acridinium esters and to a lesser extent the sulfonamides, are prone to hydrolysis to the nonluminescent carboxylic acid, the hydrolysis being accelerated at alkaline pH. The well-known problem of pseudo-base formation from attack of water at the 9-position on the ring requires a separate reaction step to regenerate the acridinium ring.

Ruthenium or osmium-containing complexes produce chemiluminescence when oxidized electrochemically in the presence of a sacrificial amine electron donor. The reaction requires a more costly and complex instrument for performing the electrochemical and light detection steps simultaneously.

U.S. Pat. Nos. 6,017,769 and 6,126,870 disclose a class of acridan compounds with heterosubstituted double bonds and a reactive linking group as chemiluminescent labels. Exemplary compounds contain a vinyl phosphate moiety. No examples of compounds bearing two sulfur substituents on the double bond were disclosed.

U.S. Pat. No. 6,858,733 discloses acridan ketenedithioacetal compounds as chemiluminescent substrates for peroxidase enzymes.

While many large molecules are used as labels, including enzymes and the photoprotein aequorin, their use suffers the disadvantage of limiting the number of labels which can be attached to the target species and having the tendency of depositing non specifically on supports and surfaces.

It remains a goal of the assay field to develop chemiluminescent labeling compounds which are small, water-soluble molecules, have high chemiluminescence efficiencies, emit the light rapidly upon reaction with simple chemical activating agents, are stable on extended storage and not subject to side reactions. The present invention provides such compounds.

Labeling Procedures. A wide variety of procedures for chemically binding labels to organic and biological molecules are described in the literature (see, for example: L. J. Kricka, Ligand-Binder Assays, Marcel Dekker, Inc., New York, 1985, pp. 15-51 and M. Z. Atassi, “Chemical Modification and Cleavage of Proteins,” Chapter 1 in Immunochemistry of Proteins, Vol. 1, Plenum Press, New York, 1977, pp. 1-161, and references therein). Antibodies and proteins are conveniently labeled by reaction of certain nucleophilic groups present in proteins (—SH, —OH, —NH₂, —COOH) with chemically reactive groups. Appropriately functionalized nucleic acids and DNA probes can also be labeled by reaction with the corresponding reactive group on a label. Many other types of molecules which can be labeled including antibodies, enzymes, protein antigens, peptides, haptens, steroids, carbohydrates, fatty acids, hormones, nucleosides and nucleotides.

Chemiluminescent Detection in Gels. A method for the detection of the enzyme alkaline phosphatase in a gel using a chemiluminescent substrate has been described (N. Theodosiou, C. Chalot, C. Ziomek, BioTechniques, 13(6), 898-901(1992)). The aforementioned U.S. Pat. Nos. 6,017,769 and 6,126,870 disclose chemiluminescent labeling compounds which can be detected as a label on a compound in an electrophoresis gel.

### SUMMARY OF THE INVENTION

It is an object of the present invention to provide methods for generating chemiluminescence from a chemiluminescent labeled compound by a simple chemical process using inexpensive, readily available reagents.

It is a further object of the present invention to provide labeling compounds for preparing chemiluminescent labeled compounds.

It is another object of the present invention to provide chemiluminescent labeled compounds.

It is also an object of the present invention to provide labeling compounds of formula I wherein one of the groups R¹-R¹¹ is a labeling substituent.

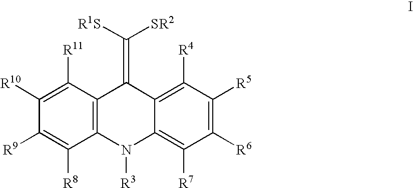

### GENERAL DESCRIPTION

Modern biomedical analyses require the ability to detect very small amounts of compounds due either to low abundance of the analyte in the sample or to limited sample quantity. In addition it must be possible to detect the quantity of the compound precisely over a very wide range of concentrations. Chemiluminescent labeling compounds and methods are disclosed herein which are suitable for these types of analyses.

The present invention relates generally to methods of generating chemiluminescence and compounds for use in these methods. The methods use acridan compounds and simple, inexpensive and readily available reagents for generating chemiluminescence therefrom. The light producing reaction can be used for a number of art-recognized purposes, including analytical methods of assay, signaling, emergency lighting and novelty items.

The present invention also involves chemiluminescent labeling compounds which can be bound to organic and biological molecules by chemical bonds or through physical interactions for the purpose of performing an assay. Reaction of the chemiluminescent compounds of the present invention according to the presently described methods produces chemiluminescence as visible light. The intensity of the resulting chemiluminescence provides a direct measure of the quantity of the chemiluminescent label and, therefore, of the labeled compound.

The present invention further involves a method for detecting a chemiluminescent labeled compound in an electrophoresis gel of the type used in separating biological molecules. Chemiluminescent labeled compounds of the present invention can be applied to a gel, separated electrophoretically and subsequently be detected in the gel without the need for transfer to a blotting membrane.

The acridan compounds useful in the methods of the invention have formula I 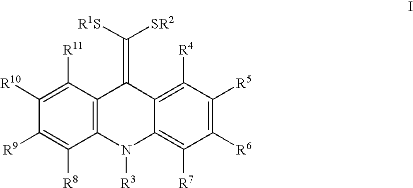
wherein at least one of the groups R¹-R¹¹ is a labeling substituent of the formula
-L-RG
wherein L is a linking group which can be a bond or another divalent or polyvalent group, RG is a reactive group which enables the chemiluminescent labeling compound to be bound to another compound, R¹ is a group removable by an acid, R² and R³ are organic groups containing from 1 to 50 non-hydrogen atoms, and each of R⁴-R¹¹ is hydrogen or a noninterfering substituent.

### DESCRIPTION OF THE PREFERRED EMBODIMENTS

Definitions:

Acid—A compound which, when added to water, causes a decrease in the pH of the resulting solution. Acid as used herein includes mineral acids, such as hydrochloric, nitric, sulfuric and perchloric, organic acids, including carboxylic acids such as oxalic, acetic and propionic, and other types of organic compounds, such as picric acid and Lewis acids, such as aluminum chloride, ferric chloride and the like.

Alkyl—A branched, straight chain or cyclic hydrocarbon group containing from 1-20 carbons. Lower alkyl as used herein refers to those alkyl groups containing up to 8 carbons.

Alkenyl—A branched, straight chain or cyclic hydrocarbon group containing at least one C—C double bond and containing from 2-20 carbons. Lower alkenyl as used herein refers to those alkenyl groups containing up to 8 carbons.

Alkynyl—A branched or straight chain hydrocarbon group containing at least one C—C triple bond and containing from 2-20 carbons. Lower alkynyl as used herein refers to those alkynyl groups containing up to 8 carbons.

Analyte—A substance the presence or amount of which is to be measured in a sample by an assay. Analytes include organic and biological molecules to which a specific binding partner having a specific binding affinity exists. Exemplary analytes include, without limitation, single stranded or double stranded DNA, RNA, DNA-RNA complexes, oligonucleotides, antibodies, antibody fragments, antibody-DNA chimeras, antigens, haptens, proteins, lectins, avidin, streptavidin and biotin. Other exemplary analytes also include drugs, hormones and pesticides.

Aryl—An aromatic ring-containing group containing 1 to 5 carbocyclic aromatic rings, which can be substituted with 1 or more substituents other than H.

Biomedical analysis—Analyses of samples of biological origin for analytes of interest. The analyses can be immunoassays, western blots, northern blots, Southern blots, DNA hybridization assays, DNA sequence analysis, colony hybridizations, gene expression analysis, high throughput drug screening, detection of infectious agents or pathogens and the like.

Glycosyl—Residues of carbohydrate groups including hexoses and pentoses and contain one or more sugar unit. Examples include fructose, galactose, glucose, glucuronate, mannose, ribose, N-acetylglucosamine and the like.

Halogen—Fluorine, chlorine, bromine or iodine atoms.

Heteroaryl—An aromatic ring-containing group containing 1 to 5 carbocyclic aromatic rings in which at least one of the ring carbon atoms is replaced with a nitrogen, oxygen or sulfur atom and which can be substituted with 1 or more substituents other than H.

Luminescent—capable of emitting light when excited to an electronic excited state. The light can be emitted either as fluorescence when decaying from a singlet excited state or as phosphorescence when decaying from a triplet excited state.

Peroxide—A compound containing an O—O bond, preferably hydrogen peroxide or a complex of hydrogen peroxide such as urea peroxide, perborate or percarbonate.

Sample—A fluid containing or suspected of containing one or more analytes to be assayed. Typical samples which are analyzed by the chemiluminescent reaction method are biological samples including body fluids such as blood, plasma, serum, urine, semen, saliva, cell lysates, tissue extracts and the like. Other types of samples include food samples and environmental samples such as soil or water.

Specific binding pair—Two substances which exhibit a mutual binding affinity. Examples include antigen-antibody, hapten-antibody or antibody-antibody pairs, complementary oligonucleotides or polynucleotides, avidin-biotin, streptavidin-biotin, hormone-receptor, lectin-carbohydrate, IgG-protein A, nucleic acid-nucleic acid binding protein and nucleic acid-anti-nucleic acid antibody.

Substituted—Refers to the replacement of at least one hydrogen atom on a group by another atom or a group having from 1 to 50 atoms selected from C, O, N, S, P, Si, B, Se, F, Cl, Br and I. It should be noted that in references to substituted groups it is intended that multiple points of substitution can be present unless indicated otherwise.

It has been discovered that chemiluminescent compounds of formula I below containing a labeling substituent undergo a reaction with certain reagents to generate chemiluminescence as a brief, intense flash of light. Use of the present compounds for detection, e.g. as labels, in chemiluminescent assays leads to highly sensitive detection of analytes. Chemiluminescent compounds of the present invention have formula I: 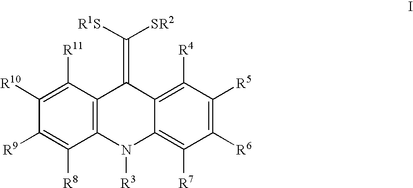
wherein R¹, R² and R³ are organic groups containing from 1 to 50 non-hydrogen atoms selected from C, N, O, S, P and halogen atoms, wherein R¹ and R² can be joined together in a ring, R⁴-R¹¹ are independently selected from hydrogen and substituents which do not interfere with the generation of chemiluminescence, and at least one of the groups R¹-R¹¹ is a labeling substituent -L-RG where L is a linking group and RG is a reactive group. The labeling substituent -L-RG is present preferably on one of R¹ or R² although it can also be present as a substituent on R³ or one of R⁴-R¹¹.

Chemiluminescent Labeling Compounds. The groups R¹ and R² can be any organic group containing from 1 to about 50 non-hydrogen atoms selected from C, N, O, S, P, Si and halogen atoms which allows light production. By the latter is meant that when a compound of formula I undergoes a reaction of the present invention, the light is produced and can involve the production of one or more chemiluminescent intermediates. R¹ and R² are preferably selected from substituted or unsubstituted alkyl, substituted or unsubstituted alkenyl, substituted or unsubstituted alkynyl, substituted or unsubstituted aryl, substituted or unsubstituted aralkyl groups of 1-20 carbon atoms. When R¹ or R² is a substituted group, it is substituted with 1-3 atoms or groups selected from carbonyl groups, carboxyl groups, tri(C₁-C₈ alkyl)silyl groups, an SO₃⁻ group, an OSO₃⁻² group, glycosyl groups, a PO₃⁻ group, an OPO₃⁻² group, halogen atoms, a hydroxyl group, a thiol group, amino groups, quaternary ammonium groups, quaternary phosphonium groups. In a preferred embodiment, R¹ or R² is preferably substituted with the labeling substituent of the formula -L-RG where L is a linking group and RG is a reactive group.

The group R³ is an organic group containing from 1 to 50 atoms non-hydrogen atoms selected from C, N, O, S, P, Si and halogen atoms in addition to the necessary number of H atoms required satisfy the valencies of the atoms in the group. More preferably R³ contains from 1 to 20 non-hydrogen atoms. The organic group is preferably selected from the group consisting of alkyl, substituted alkyl, substituted or unsubstituted alkenyl, substituted or unsubstituted alkynyl, substituted or unsubstituted aryl, substituted or unsubstituted aralkyl groups of 1-20 carbon atoms. More preferred groups for R³ include substituted or unsubstituted C₁-C₄ alkyl groups, phenyl, substituted or unsubstituted benzyl groups, alkoxyalkyl, carboxyalkyl and alkylsulfonic acid groups. The group R³ can be joined to either R⁷ or R⁸ to complete a 5 or 6-membered ring. In one embodiment, R³ is substituted with the labeling substituent of the formula -L-RG.

In the compounds of formula I, the groups R⁴-R¹¹ each are independently H or a substituent group which permits the light to be produced and generally contain from 1 to 50 atoms selected from C, N, O, S, P, Si and halogen atoms. Representative substituent groups which can be present include, without limitation, alkyl, substituted alkyl, aryl, substituted aryl, aralkyl, alkenyl, alkynyl, alkoxy, aryloxy, halogen, amino, substituted amino, carboxyl, carboalkoxy, carboxamide, cyano, and sulfonate groups. Pairs of adjacent groups, e.g. R⁴-R⁵ or R⁵-R⁶, can be joined together to form a carbocyclic or heterocyclic ring system comprising at least one 5 or 6-membered ring which is fused to the ring to which the two groups are attached. Such fused heterocyclic rings can contain N, O or S atoms and can contain ring substituents other than H such as those mentioned above. One or more of the groups R⁴-R¹¹ can be a labeling substituent of the formula -L-RG. It is preferred that R⁴ to R¹¹ are selected from hydrogen, halogen and alkoxy groups such as methoxy, ethoxy, t-butoxy and the like. A preferred group of compounds has one of R⁵, R⁶, R⁹ or R¹⁰ as a halogen and the other of R⁴ to R¹¹ are hydrogen atoms.

Substituent groups can be incorporated in various quantities and at selected ring or chain positions in the acridan ring in order to modify the properties of the compound or to provide for convenience of synthesis. Such properties include, e.g. chemiluminescence quantum yield, rate of reaction with the enzyme, maximum light intensity, duration of light emission, wavelength of light emission and solubility in the reaction medium. Specific substituents and their effects are illustrated in the specific examples below, which, however, are not to be considered limiting the scope of the invention in any way.

For synthetic expediency compounds of formula I desirably have each of R⁴ to R¹¹ as a hydrogen atom.

Linking group (L). The linking group can be a bond, an atom, divalent groups and polyvalent groups, or a straight, or branched chain of atoms some of which can be part of a ring structure. The substituent usually contains from 1 to about 50 non-hydrogen atoms, more usually from 1 to about 30 non-hydrogen atoms. Atoms comprising the chain are selected from C, O, N, S, P, Si, B, and Se atoms, preferably from C, O, N, P and S atoms. Halogen atoms can be present as substituents on the chain or ring. Typical functional groups comprising the linking substituent include alkylene, arylene, alkenylene, ether, peroxide, carbonyl as a ketone, ester, carbonate ester, thioester, or amide group, amine, amidine, carbamate, urea, imine, imide, imidate, carbodiimide, hydrazine, diazo, phosphodiester, phosphotriester, phosphonate ester, thioether, disulfide, sulfoxide, sulfone, sulfonate ester, sulfate ester, and thiourea groups.

Reactive group. The reactive group RG is an atom or group whose presence facilitates bonding to another molecule by covalent attachment or physical forces. In some embodiments, attachment of a chemiluminescent labeling compound of the present invention to another compound will involve loss of one or more atoms from the reactive group for example when the reactive group is a leaving group such as a halogen atom or a tosylate group and the chemiluminescent labeling compound is covalently attached to another compound by a nucleophilic displacement reaction. In other embodiments, attachment of a chemiluminescent labeling compound to another compound by covalent bond formation will involve reorganization of bonds within the reactive group as occurs in an addition reaction such as a Michael addition or when the reactive group is an isocyanate or isothiocyanate group. In still other embodiments, attachment will not involve covalent bond formation, but rather physical forces in which case the reactive group remains unaltered. By physical forces is meant attractive forces such as hydrogen bonding, electrostatic or ionic attraction, hydrophobic attraction such as base stacking, and specific affinity interactions such as biotin-streptavidin, antigen-antibody and nucleotide-nucleotide interactions.

TABLE 1
Reactive Groups for Chemical Binding of Labels to
Organic and Biological Molecules
a.) Amine reactive groups.

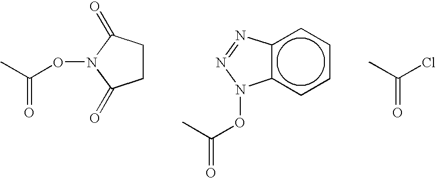

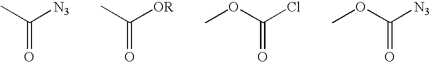

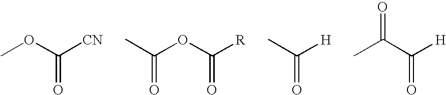

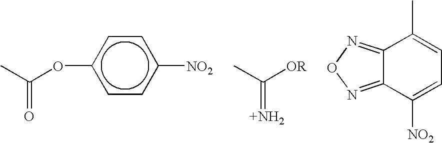

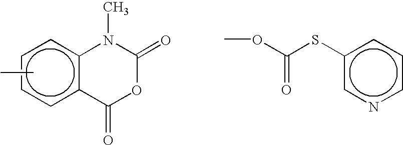

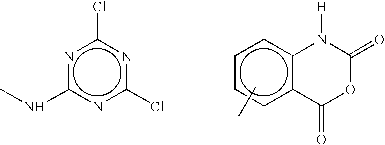

| | | | |
|---|---|---|---|
| —N═C═S | —SO₂Cl | —N═C═O | —SO₂CH₂CF₃ |

b.) Thiol reactive groups.

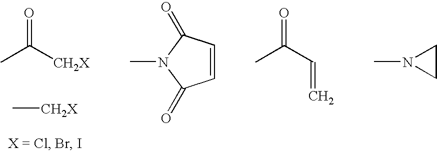

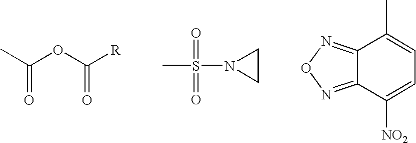

| | |
|---|---|
| —S—S —R | 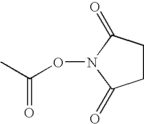 |

3) Carboxylic acid reactive groups.

| | | | |
|---|---|---|---|
| —NH₂ | —OH | —NHNH₂ | 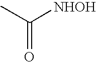 |

4) Hydroxyl reactive groups.

| | | |
|---|---|---|
| 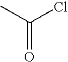 | 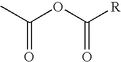 | —N═C═S |
| —SO₂Cl | —N═C═O | —SO₂CH₂CF₃ |

5) Aldehyde/ketone reactive groups.

| | | |
|---|---|---|
| —NH₂ | —ONH₂ | —NHNH₂ |

6) Other reactive group pairs.

| | |
|---|---|
| 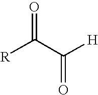 | 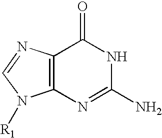 |
| R—N₃ | R—C≡CH |

Preferred reactive groups include OH, NH₂, ONH₂, NHNH₂, COOH, SO₂CH₂CF₃, N-hydroxysuccinimide ester, N-hydroxy-succinimide ether and maleimide groups.

Bifunctional coupling reagents can also be used to couple labels to organic and biological molecules with moderately reactive groups (see L. J. Kricka, Ligand-Binder Assays, Marcel Dekker, Inc., New York, 1985, pp. 18-20, Table 2.2 and T. H Ji, “Bifunctional Reagents,” Methods in Enzymology, 91, 580-609 (1983)). There are two types of bifunctional reagents, those which become incorporated into the final structure and those which do not and serve only to couple the two reactants.

A preferred group of compounds have formula II wherein each of R⁴ to R¹¹ are hydrogen. The groups R¹, R² and R³ are as defined above.

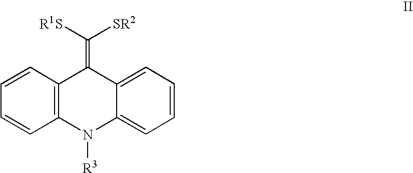

Preferred labeling compounds have formulas V or VI.

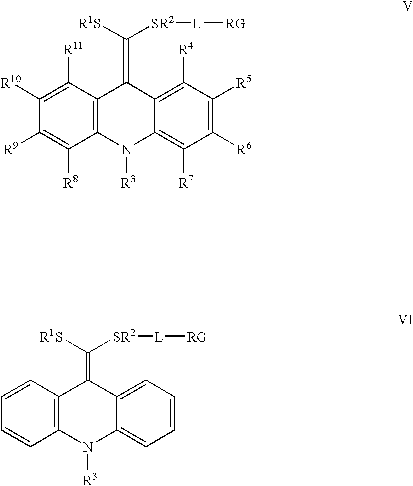

In another aspect, the invention relates to chemiluminescent labeled compounds. By this is meant conjugates of a compound which is to be detected and at least one moiety comprising a chemiluminescent labeling compound of formula I. When preparing a conjugate using a labeling compound of formula V: 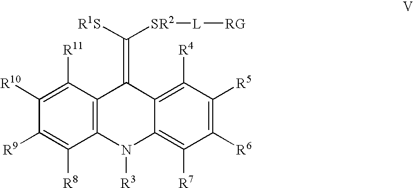
the compound to be labeled with the chemiluminescent label will become attached by means of the reactive group RG of V. The attachment may result in the displacement of a portion of the reactive group RG. For example when an N-hydroxysuccinimide ester is RG, the N-hydroxysuccinimide portion is lost in forming the link. In other cases, RG is intact as for example when it is a maleimide group reacting with an —SH group on a compound being labeled or an isocyanate reacting with an amine or —OH group. In still other cases, the entire RG is lost in forming the link; an example would be when RG is a leaving group such as a halide, azide, or p-toluenesulfonate.

When preparing the chemiluminescent labeled compound, a molar excess of the chemiluminescent labeling compound is typically used although it is not necessary. The chemiluminescent labeling compound is preferably used in at least 5-fold molar excess to the compound to be labeled and usually in at least a 1-fold molar ratio. The chemiluminescent labeled compound may be labeled with one labeling group or multiple copies of the group. In general it is desirable to incorporate multiple labels to increase the amount of signal which can be generated.

Synthetic Methods. Compounds of formula I can be prepared by various methods. In a preferred method compound I can be prepared by reacting the enolate of a dithioester with a reagent of the formula R¹-LG where LG represents a leaving group as exemplified by the scheme below.

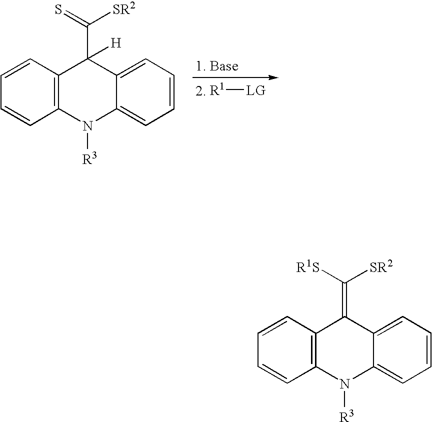

Typical leaving groups include halogens, such as chloride, bromide and iodide, sulfonates such as methanesulfonate and p-toluenesulfonate and trifluoromethanesulfonate, carboxylates such as acetate and benzoate, sulfates such as methosulfate, and other groups such as imidazole, triazole and tetrazole, maleimide, succinimidoxy groups.

Methods of preparing compounds of formula I also include nucleophilic addition of a lithiosilane compound or a phosphorus ylide to a suitable carbonyl compound according to the two schemes below (F. A. Carey, A. S. Court, J. Org. Chem., 37, 1926-29, (1972)).

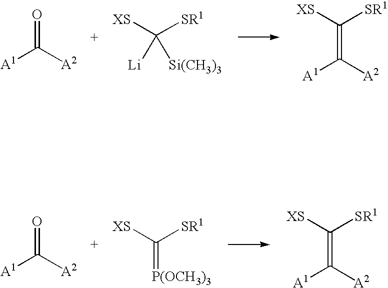

In another method, an ester is converted to a ketene-dithioacetal by reaction with a bis(dialkylaluminum)-dithiol reagent as disclosed in E. J. Corey and A. P. Kozikowski, Tetrahedron Lett., 925-8 (1975) and shown below.

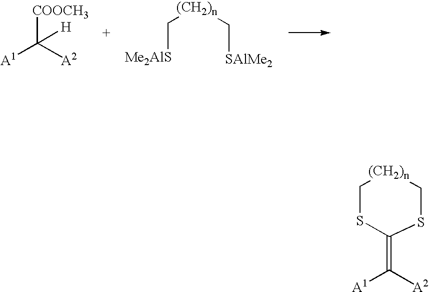

In yet another method, an anion of an active methylene group is reacted with CS and the dithiocarboxylate is reacted with a reagent R¹-LG containing the R¹ group to form a dithioester. An example of the latter methodology is disclosed in I. Shahak and Y. Sasson, Tetrahedron Lett., 4207-10 (1973). The dithioester is converted to the enolate and reacted with a reagent of the formula X-LG.

Methods of preparing chemiluminescent labeling compounds generally involve preparing a precursor compound of formula I and subjecting it to one or more additional reactions, generally known to the skilled artisan, to provide a labeling substituent appended to one of the groups R¹ to R¹¹, preferably R¹ or R². Numerous examples are provided below to illustrate the general principle.

Methods of Generating Chemiluminescence. The present invention relates to methods for producing chemiluminescence comprising subjecting a labeled substance to conditions for producing chemiluminescence from the label moiety. In particular the methods are applied in the service of assay methods for detecting an analyte in a sample.

In one embodiment of the present methods for producing chemiluminescence, a compound of formula I or a substance labeled with a compound of formula I undergoes a reaction comprising the steps of:

a) contacting the compound of formula I with an acid to form a first reaction product; and

b) contacting the first reaction product with a sufficient quantity of a base to provide a basic environment, at least one of the steps including providing an oxidant for reaction, wherein a second reaction product is formed and the light is produced in the basic environment. Light intensity reaches a maximum level rapidly, often within a second or less, at room temperature when the reaction is conducted at alkaline pH.

The acid used in the first step must be capable of providing a low pH environment, at least below about 3 and preferably not greater than 1. Mineral acids are preferred because of their low cost and high acidity. In some instances, oxidizing mineral acids, e.g., nitric acid may be preferred. Acids will typically be used at a concentration in the range 0.001 M to 1 M.

The oxidant can be a peroxide or alkyl hydroperoxide. Preferred peroxides include hydrogen peroxide, urea peroxide, persulfate and perborate salts. The oxidant can also be a metal oxide such as CrO₃, MnO₂ or an anionic complex such as periodate IO₄⁻ or permanganate MnO₄⁻, or a metal peroxide such as Na₂O₂. Other oxidants include heme or hemoglobin. The acid can also function, in part, as an oxidant, as for example, when the acid is nitric acid. The choice of whether it is preferred to combine the oxidant with the acid in the first step or the base in the second step is influenced by the choice of the acid and the stability and reactivity of the oxidant in the base. In general, it may be advantageous to combine the oxidant with the base when the acid is an oxidizing acid. In other cases, it may be advantageous to combine the oxidant with the acid.

Basic compounds useful in the practice of the present invention comprise compounds which, when added to water causes an increase in the pH of the resulting solution. This includes hydroxide salts, such as sodium, potassium or lithium hydroxide, ammonium hydroxide and tetraalkylammonium hydroxide, carbonates and basic metal oxides. The use of organic bases is also contemplated. The preferred bases are the alkali metal hydroxides. The reaction is typically performed at a temperature between 5° C. and 50° C., preferably between 20° C. and 40° C., and usually at ambient temperature. The reaction of the present invention is carried out in aqueous solution which may be in contact with the surface of a solid support such as a bead, tube, membrane or microwell plate coated with peroxidase. In some assay formats, it may be desirable to perform assay steps involving binding reactions in buffer solution. The acid must be used in a quantity and concentration sufficient to overcome the buffering capacity and lower the solution pH to not more than 3 and preferably about 1 or lower.

In another method of producing chemiluminescence from a labeling compound of formula I or a substance labeled with a labeling compound of formula I, the compound is reacted with singlet oxygen, ¹O₂.

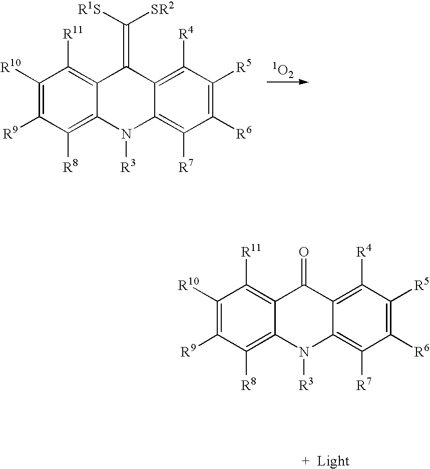

In another method of producing chemiluminescence from a labeling compound of formula I or a substance labeled with a labeling compound of formula I, the compound is reacted with a peroxidase and a peroxide. In contrast to the methods disclosed in Applicants' U.S. Pat. No. 6,858,733, the peroxidase will be present in excess and the labeled compound will be the limiting reagent. This difference manifests in the chemiluminescence signal being produced as a brief flash of light, typically lasting only a few seconds or less.

In another method of producing chemiluminescence from a labeling compound of formula I or a substance labeled with a labeling compound of formula I, the compound is reacted with hydrogen peroxide and a transition metal ion. Preferred transition metal ions include Fe⁺², Fe⁺³, Co⁺², Co⁺³, Cu⁺², Ni⁺², Cr⁺³, Zn⁺² and Mn⁺². The metal ion can be present as the free ion or as a complex with an organic ligand.

Chemiluminescent Assay Methods. Another aspect of the present invention is the use of the chemiluminescent reaction in a method to detect the presence, location or amount of an analyte labeled with a chemiluminescent label of the invention, comprising providing a substance labeled with a compound of formula I, generating the light by the chemiluminescent reaction with the labeled compound, detecting the light produced and, if quantitation is desired, relating the amount of light produced to the amount of the analyte. The relationship between light intensity and amount of analyte can be easily discerned by constructing a calibration curve with known amounts of the chemiluminescent compound. The overall chemiluminescent reaction can be illustrated by the reaction below.

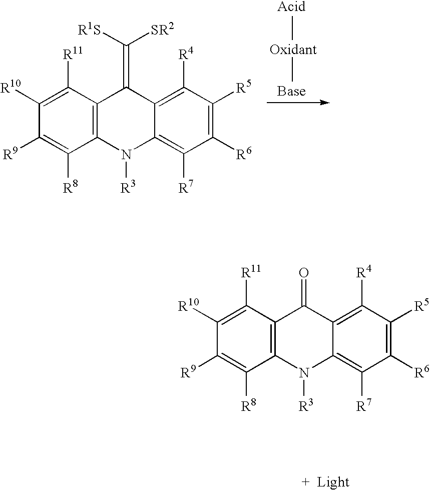

Analytes. Substances which can be assayed by employing the present chemiluminescent methods in an assay procedure include various classes of organic and biological molecules. In one embodiment such assays can involve the use of a specific binding reaction between at least one pair of specific binding partners. At least one of the specific binding partners is labeled with a compound of formula I in the manner described above. Exemplary analytes include drugs, hormones, pesticides, pesticide metabolites, DNA, RNA, oligonucleotides, antibodies, antibody fragments, antibody-DNA chimeras, antigens, haptens, proteins, carbohydrates, lectins, receptors, avidin, streptavidin and biotin. Exemplary binding partners include antigen-antibody, hapten-antibody or antibody-antibody pairs, complementary oligonucleotides or polynucleotides, avidin-biotin, streptavidin-biotin, hormone-receptor, lectin-carbohydrate, IgG-protein A, nucleic acid-nucleic acid binding protein and nucleic acid-anti-nucleic acid antibody.

In another embodiment a labeled compound comprising a substance labeled with a compound of formula I can be a tracer compound. Such compounds may find use in examining the fate or spatial distribution of a substance in the environment or in a bacterial, plant or animal organism.

An exemplary use is in an immunoassay. The analyte hapten, antigen or antibody is assayed by detecting the presence or amount of a chemiluminescent-labeled specific binding partner for the analyte or a labeled analog of the analyte. Various assay formats and the protocols for performing the immunochemical steps are well known in the art. These assays fall broadly into two categories. Competitive assays feature an immunological binding of a specific antibody with the analyte and an analyte analog, e.g. a detectably labeled analyte molecule. Sandwich assays result by the sequential or simultaneous binding of two antibodies, one of which is detectably labeled, with the analyte. The detectably labeled binding pair so formed can be assayed with the compounds and methods of the present invention. Measurement can be performed with labeled species attached to a solid surface or support including beads, tubes, microwells, magnetic particles, latex particles, silica particles, test strips, membranes and filters such as are in common use in the art.

Another exemplary use is the detection of nucleic acids by the use of labeled nucleic acid probes. Methods for analysis and chemiluminescent detection of nucleic acids using labeled probes, for example, solution hybridization assays, DNA detection in Southern blotting, RNA by Northern blotting, DNA sequencing, DNA fingerprinting, colony hybridizations and plaque lifts are all well established techniques. The label can be present as a direct conjugate with a probe oligonucleotide or capture oligonucleotide or it can be incorporated through indirect linking means using art-known methods. Examples of indirect linking means include using hapten-labeled oligonucleotides and labeled anti-hapten antibodies or biotinylated oligonucleotides and labeled avidin or labeled streptavidin. Such nucleic acid assays can be performed on a blotting membrane or in solution using oligonucleotides attached to solid surfaces including beads, tubes, microwells, magnetic particles, microarrays or test strips as are known in the art.

Use of the present chemiluminescent reaction for detection of labeled analytes, such as nucleic acids, proteins or antibodies, provides an advantage over most other chemiluminescent labeling methods. It has been found that the chemiluminescent-labeled analyte can undergo electrophoresis and be directly detected in gels such as acrylamide and agarose. The labeled analyte is not destroyed or triggered at the electrical potential and currents employed in the process as would be expected based on the prior art. This technique represents a significant advance in detection methodology by removing the need for a membrane transfer step and should be particularly well suited for detection of DNA sequencing ladders. In comparison to the labeling compounds disclosed in Applicants' U.S. Pat. Nos. 6,017,769 and 6,126,870, the present labeling compounds and labeled compounds present better stability. The present ketene dithioacetal labels (carbon-carbon double bond with two sulfur atoms at the terminal carbon) are markedly more stable in aqueous or protic solutions than the compounds with a sulfur atom and a phosphate group on the terminal carbon. Moreover, the absence of a phosphate group in the present labels provides increased flexibility in synthesis, particularly in preparing a compound bearing the useful N-hydroxysuccinimide group.

Another exemplary use is the immunological detection of proteins in gels or by the technique of Western blotting. A sample containing a protein of interest as the analyte is subject to electrophoretic separation. The separated proteins are either detected directly in the gel or transferred to a blotting membrane such as a nitrocellulose or PVDF membrane by capillary action or with the aid of an electric field. Transferred protein is detected with either a labeled primary antibody or a specific primary antibody and a labeled secondary antibody which recognizes and binds to the primary antibody. Quantitative determination of the label reflects the presence of the analyte protein. To adapt the methods of the present invention for Western blotting, antibody is labeled with a chemiluminescent labeling compound of the present invention. Variations on this technique such as using biotinylated antibodies and chemiluminescent labeled avidin are considered within the scope of the invention.

Light emitted by the present method can be detected by any suitable mean, including luminometers, x-ray film, high speed photographic film, a CCD camera or visually. Choice of the detection device will be governed by the application and considerations of cost, convenience, spectral sensitivity and need for a permanent record.

Multi-analyte assays can be performed using two or more distinguishable chemiluminescent labels concurrently to label different analytes. Appropriately chosen chemiluminescent labels may be independently detected on the basis of different emission wavelengths. Alternatively two or more different labels may be distinguishable by the time required to emit the light. Methods for chemiluminescent multi-analyte assays are disclosed in U.S. Pat. No. 5,656,207, the disclosure of which is incorporated herein by reference. Multi-analyte assays can also include detecting multiple regions of the same analyte, such as two different regions of a nucleic acid or two epitopes of an antigen. This type of assay is useful, for example, for detecting gene juxtapositions or for providing increased specificity of detection.

The use of surfactants as additives in the present chemiluminescent reactions is advantageous and can lead to an improvement in analytical sensitivity. Nonionic surfactants useful in the practice of the present invention include by way of example polyoxyethylenated alkylphenols, polyoxyethylenated alcohols, polyoxy-ethylenated ethers and polyoxyethylenated sorbitol esters. Cationic surfactants, including quaternary ammonium salt compounds such as CTAB, are advantageous for use in increasing the level of chemiluminescence emitted.

In a further embodiment, fluorescent energy acceptors can be employed to shift the maximum emission to longer wavelengths (red-shifting) and/or to increase the quantity of luminescence emitted. Fluorescers can be covalently linked to a compound of formula I or, alternatively, can be added to the reaction solution as separate species, or linked to a polymer or electrostatically associated with a micelle or polymer.

### EXAMPLES

### Example 1

### Synthesis of Compound 1

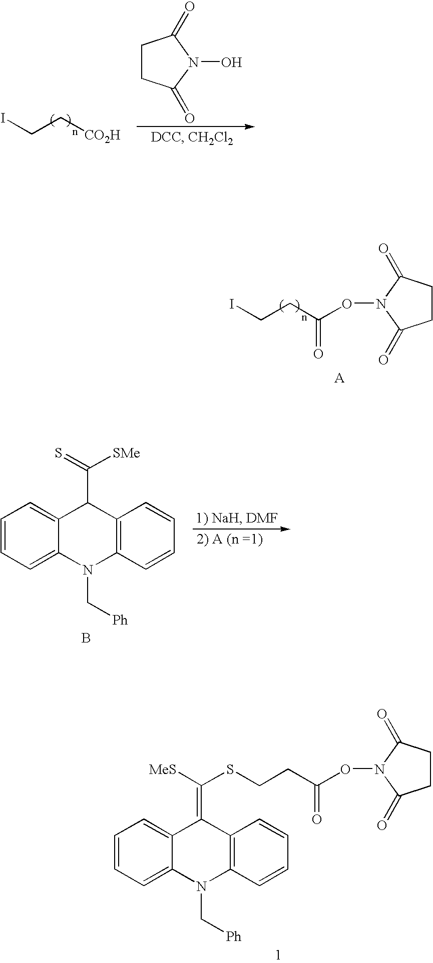

The iodocarboxylate NHS ester was synthesized by reacting the iodocarboxylic acids with N-hydroxy-succinimide using DCC as the coupling reagent.

To a solution of dithioester B (1.808 g, 5.00 mmol) in anhydrous DMF (50 mL) was added NaH (60% in mineral oil, 0.200 g, 5.00 mmol) under argon. After 4 h at room temperature NHS 3-iodopropionate A (1.485 g, 5.00 mmol) was added and the resulting mixture was stirred overnight. DMF was removed in vacuo. Column chromatography with CH₂Cl₂/EtOAc (40:1) afforded 1.770 g of 1 as a yellow solid (yield 67%). ¹H NMR (300 MHz, CDCl₃): δ2.30 (s, 3H), 2.74 (t, 2H), 2.83 (s, 4H), 3.01 (t, 2H), 5.31 (s, 2H), 6.88(t, 2H), 7.07 (m, 2H), 7.11-7.18 (m, 3H), 7.27 (m, 4H), 7.82 (dd, 1H), 7.89 (dd, 1H) ppm.

### Example 2

### Synthesis of Compound 2

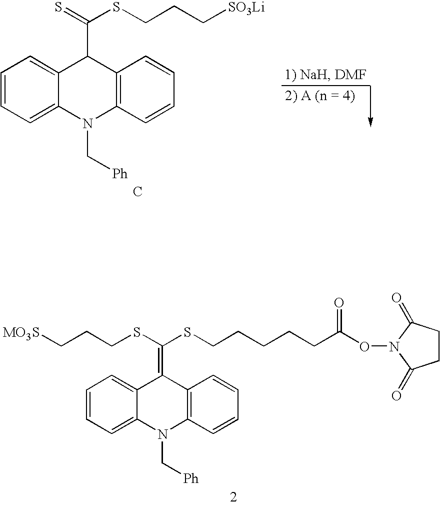

A mixture of dithioester C (0.692 g, 1.50 mmol) and NaH (60% in mineral oil, 0.060 g, 1.50 mmol) in anhydrous DMF (20 mL) was stirred under argon at room temperature for 4 hours, resulting a slightly cloudy solution. NHS 6-iodohexanoate A (0.661 g, 1.95 mmol) was then added in DMF (5 mL). After 16 h, DMF was removed in vacuo. To the residue was added 10 mL of acetone followed by 20 mL of ether. The supernatant was decanted. The precipitate was washed three times following the same procedures. After drying under vacuum, 1.200 g of 2 was obtained as a yellow solid. ¹H NMR (300 MHz, CD₃OD):δ1.15 (m, 2H), 1.33-1.47 (m, 4H), 2.01 (p, 2H), 2.38 (t, 2H), 2.67 (t, 2H), 2.75 (t, 2H), 2.82 (s, 4H), 2.88 (t, 2H), 5.32 (s, 2H), 6.88-6.93 (m, 2H), 7.00 (t, 2H), 7.08-7.28 (m, 7H), 7.83 (d, 1H), 7.92 (d, 1H) ppm.

### Example 3

### Synthesis of Compounds 3 and 4

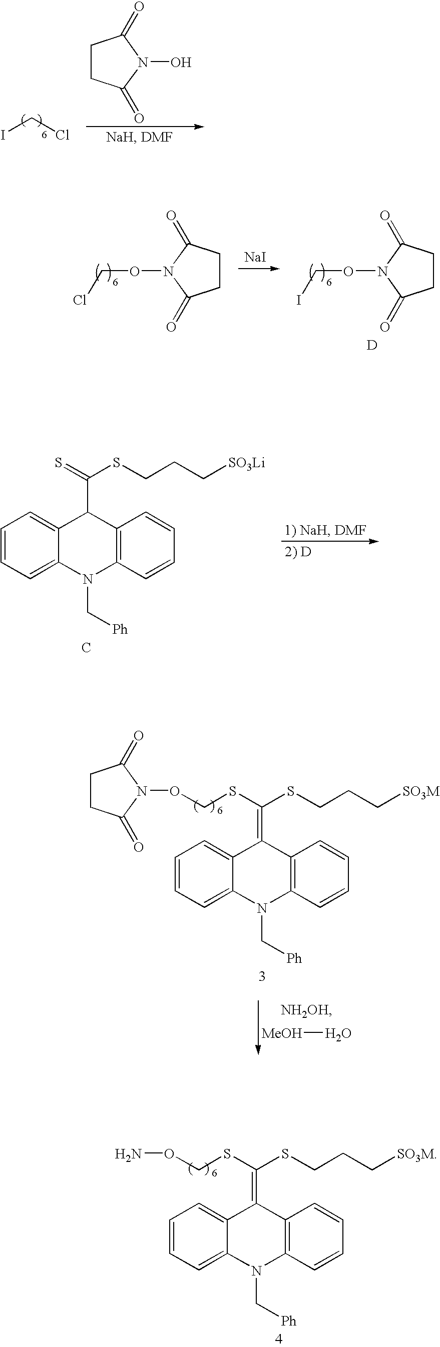

A mixture of dithioester C (1.00 g, 2.10 mmol) and NaH (60% in mineral oil, 0.087 g, 2.16 mmol) in anhydrous DMF (20 mL) was stirred under argon at room temperature for 4 hours, resulting in a slightly cloudy solution. N-6-iodohexoxysuccinimide D (0.82 g, 2.52 mmol) was then added in DMF (5 mL). The mixture was stirred over night after which DMF was removed in vacuo. The residue was washed four times with 30 mL of ether giving 1.35 g of 3.

Compound 3 (0.25 g) was dissolved in 5 mL of methanol to which was added 5.0 mL of 50% aq. NH₂OH. After stirring the solution for 2 days, the solvents were evaporated under vacuum. The residue was washed with 6×20 mL of ether giving 0.21 g of 4. ¹H NMR (300 MHz, CD₃OD):δ1.14 (m, 4H), 1.40 (m, 4H), 1.94 (p, 2H), 2.65-2.71 (m, 4H), 2.84 (t, 2H), 3.55 (t, 2H), 5.31 (s, 2H), 6.88 (d, 2H), 6.98 (q, 2H), 7.10 (m, 4H), 7.12-7.27 (m, 3H), 7.85 (t, 2H) ppm.

### Example 4

### Synthesis of Compounds 5 and 6

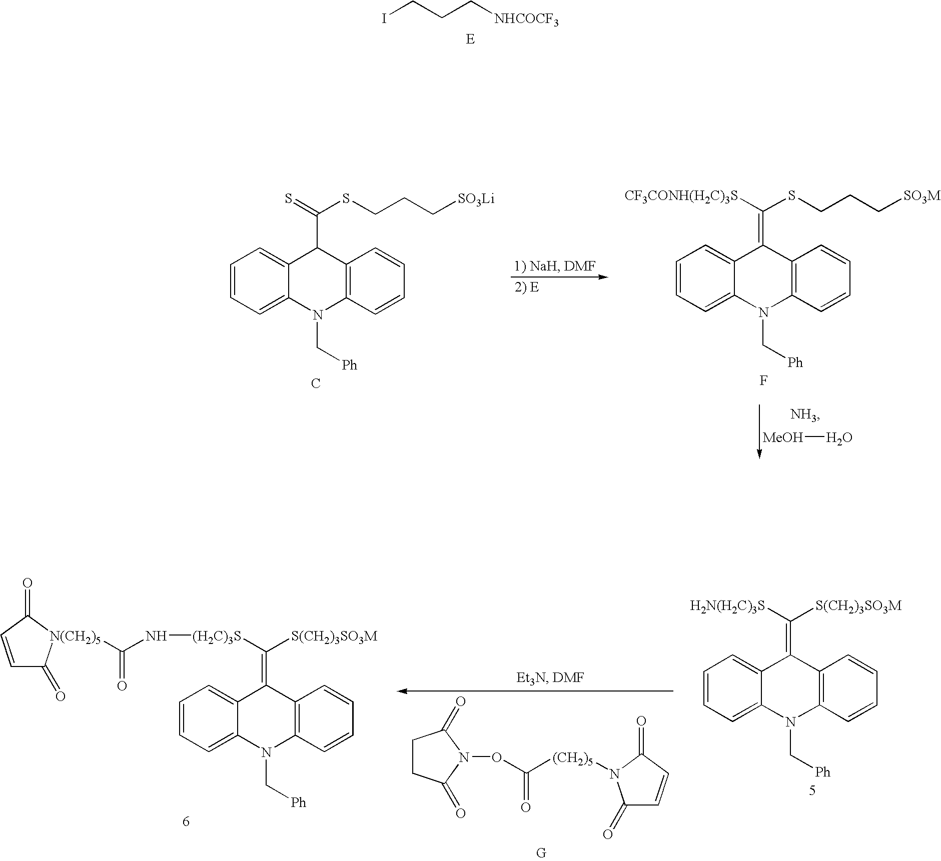

A mixture of dithioester C (1.32 g, 2.78 mmol) and NaH (60% in mineral oil, 0.114 g, 2.86 mmol) in 30 mL of anh. DMF was stirred under argon at room temperature for 4 hours. Compound E (1.014 g, 3.61 mmol) was then added in 10 mL of DMF. The mixture was stirred over night after which DMF was removed in vacuo. The residue was washed three times with 20 mL of ether giving 2.10 g of F.

Compound F (2.25 g) was dissolved in a mixture of 15 mL of 7 N NH₃ in MeOH and 10 mL of 28% aqueous ammonia solution. After 3 days of stirring, solvents were removed under vacuum. The residue was washed with ether (3×50 mL) and recrystallized with H₂O/2-propanol, giving 1.20 g of 5.

To a suspension of 5 (0.300 g, 0.563 mmol) in 9.0 mL of dry DMF was added 1.20 mL of triethylamine. The mixture was stirred for 5 min, giving a slightly cloudy solution. To this was added 6-maleimidohexanoic NHS ester (G 0.260 g, 0.843 mmol). A clear solution as resulted in 5 min. After 16 hrs, DMF was removed under vacuum. The residue was washed with ether (4×30 mL), then dissolved in MeOH (2 mL) and precipitated with ether (50 mL). A 0.400 g yield of 6 was obtained as a yellowish foam-like solid. ¹H NMR (400 MHz, CD₃OD): δ 1.26 (t, 11H), 1.49-1.58 (m, 6H), 1.90 (p, 2H), 2.08 (t, 2H), 2.68 (m, 4H), 2.80 (t, 2H), 3.00 (t, 2H), 3.15 (q, 6H), 3.42 (t, 2H), 5.28 (s, 2H), 6.73 (s, 2H), 6.85 (d, 2H), 6.96 (m, 2H), 7.07 (m, 4H), 7.18-7.25 (m, 3H), 7.83 (m, 2H) ppm.

### Example 5

### Additional Labeling Compounds 7-12

The preparation of other exemplary labeling compounds listed below was disclosed in U.S. Pat. No. 6,858,733.

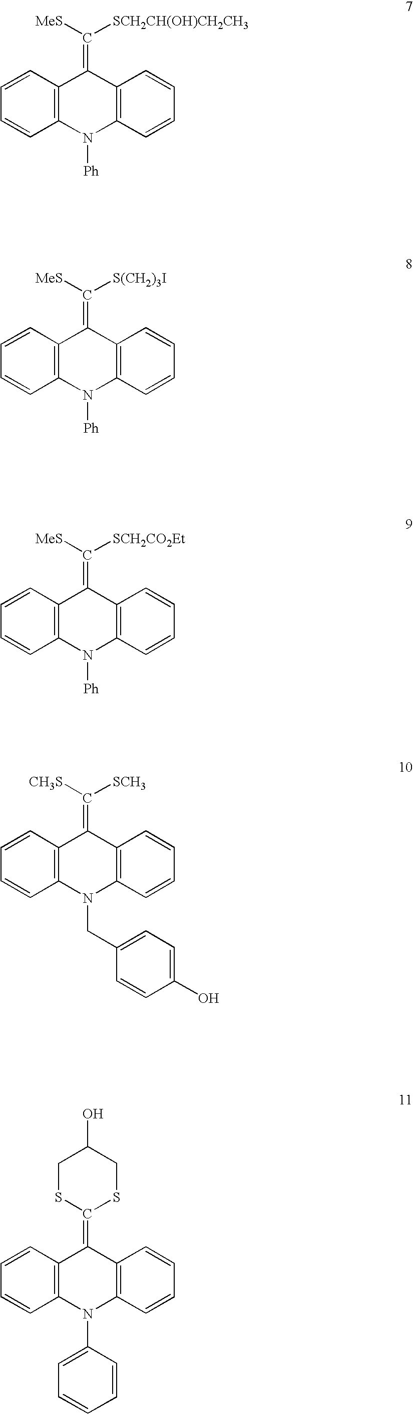

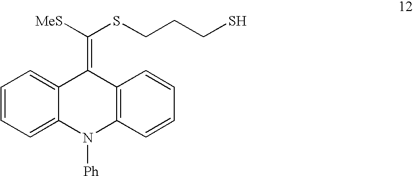

The foregoing description and examples are illustrative only and not to be considered as restrictive. It is recognized that modifications of the specific compounds and methods not specifically disclosed can be made without departing from the spirit and scope of the present invention. The scope of the invention is limited only by the appended claims.

1. A method for nonelectrochemically producing chemiluminescence comprising subjecting a substance bearing a chemiluminescent label moiety to conditions for producing chemiluminescence from the label moiety wherein the label is provided by reacting the substance with a compound of the formula I: 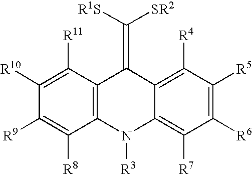
wherein R¹, R² and R³ are organic groups containing from 1 to 50 non-hydrogen atoms selected from C, N, O, S, P and halogen atoms, wherein each of R¹, R², and R³ is selected from substituted or unsubstituted alkyl, substituted or unsubstituted alkenyl, substituted or unsubstituted alkynyl, substituted or unsubstituted aryl, substituted or unsubstituted aralkyl groups of 1-20 carbon atoms, wherein R¹ and R² can be joined together in a ring, wherein R¹ and R² are not removable by an acid, R⁴-R¹¹ are independently selected from hydrogen and substituents which do not interfere with the generation of chemiluminescence, and at least one of the groups R¹-R¹¹ is a labeling substituent -L-RG where L is a linking group and RG is a reactive group.

2. The method of claim 1 wherein the labeling substituent -L-RG is present as a substituent on R¹ or R².

3. The method of claim 1 wherein the labeling substituent -L-RG is present as a substituent on R³.

4. The method of claim 1 wherein each of R⁴-R¹¹ is a hydrogen atom.

5. (canceled)

6. The method of claim 5 wherein the labeling substituent -L-RG is present as a substituent on R¹, wherein R² is alkyl substituted with an SO₃⁻ group, and wherein R³ is selected from substituted or unsubstituted C₁-C₄ alkyl groups, phenyl, substituted or unsubstituted benzyl groups.

7. The method of claim 1 wherein L is selected from a bond, divalent groups and polyvalent groups containing from 1 to 30 non-hydrogen atoms selected from C, N, O, P and S atoms.

8. The method of claim 6 wherein L is selected from a bond, divalent groups and polyvalent groups containing from 1 to 30 non-hydrogen atoms selected from C, N, O, P and S atoms.

9. The method of claim 1 wherein RG is a reactive group selected from carboxyl, carboxyl ester, acid anhydride, acid chloride, acyl azide, aldehyde, chloroformate, amine, hydroxyl, hydrazine, hydrazide, hydroxylamine, isocyanate, isothiocyanate, sulfonyl chloride, SO₂CH₂CF₃, tosyl, maleimide, N-hydroxysuccinimide ester, aziridine, disulfide, azide, halogen, 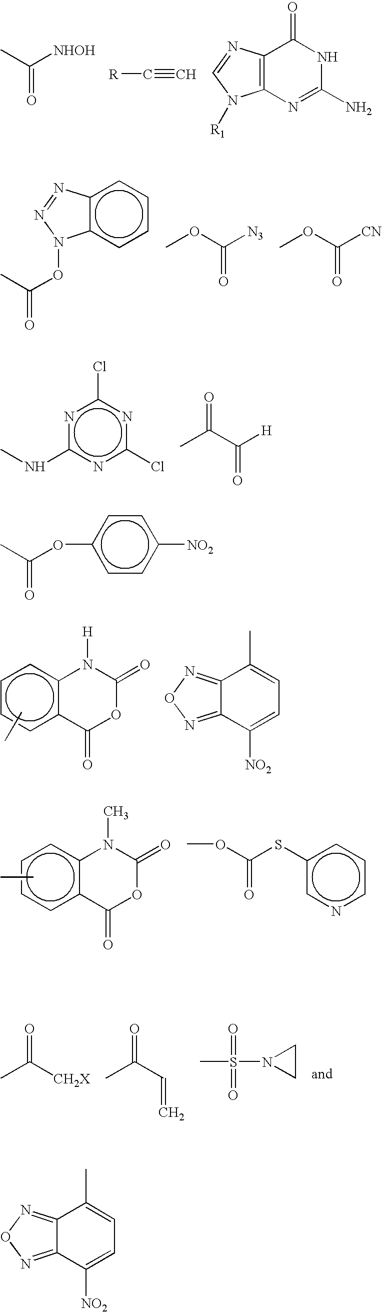
wherein X is selected from chlorine, bromine and iodine.

10. The method of claim 1 wherein the reactive group is selected from OH, NH₂, COOH, SO₂CH₂CF₃, N-hydroxy-succinimide ester, N-hydroxysuccinimide ether, and maleimide groups.

11. The method of claim 1 wherein the labeled substance is an analyte or a specific binding pair member.

12. The method of claim 11 wherein the analyte is selected from drugs, hormones, pesticides, pesticide metabolites, DNA, RNA, oligonucleotides, antibodies, and antigens.

13. The method of claim 11 wherein the specific binding pair member is selected from antigens, antibodies, haptens, oligonucleotides, polynucleotides, avidin, streptavidin, hormones, receptors, lectins, carbohydrates, IgG, protein A, and nucleic acid binding proteins.

14. The method of claim 1 used to detect the presence, location or amount of an analyte comprising:
a) providing a substance labeled with a compound of formula I;
b) generating the chemiluminescence by the chemiluminescent reaction with the labeled compound;
c) detecting the chemiluminescence produced; and
d) relating the light produced to the presence, location or amount of the analyte.

15. The method of claim 1 wherein the labeled substance is a tracer compound.

16. The method of claim 1 wherein the labeled substance comprises more than one of the same chemiluminescent label moiety.

17. The method of claim 1 wherein the conditions for producing chemiluminescence from the label moiety comprise reacting the labeled substance with singlet oxygen.

18. The method of claim 1 wherein the conditions for producing chemiluminescence from the label moiety comprise reacting the labeled substance with a peroxidase and a peroxide.

19. The method of claim 1 wherein the conditions for producing chemiluminescence from the label moiety comprise reacting the labeled substance with hydrogen peroxide and either a transition metal ion or a complex of a transition metal ion with an organic ligand.

20. The method of claim 1 wherein the conditions for producing chemiluminescence from the label moiety comprise:
a) reacting the labeled substance with an acid to form a first reaction product; and
b) contacting the first reaction product with a sufficient quantity of a base to provide a basic environment, at least one of the steps including providing an oxidant for reaction, wherein the chemiluminescence is produced in the basic environment.

21. The method of claim 20 wherein the acid is a mineral acid, the base is selected from hydroxide salts, carbonates, basic metal oxides and organic bases, and the oxidant is selected from hydrogen peroxide, alkyl hydroperoxide, complexes of hydrogen peroxide, permanganate, periodate, metal oxides and metal peroxides.
